(19) United States
(12) Patent Application Publication
(10) Pub. No.: US 2025/0120722 A1
(43) Pub. Date: Apr. 17, 2025

(54) MEDICAL CLIP, FORMING TOOL, FORMING MACHINE, AND METHOD FOR PRODUCING A MEDICAL CLIP

(71) Applicant: Aesculap AG, Tuttlingen (DE)

(72) Inventors: Jens Beger, Tuttlingen (DE); Alexander Happle, Huefingen (DE); Mike Popp, Chemnitz (DE); Mike Lahl, Chemnitz (DE)

(21) Appl. No.: 18/664,794

(22) Filed: May 15, 2024

Related U.S. Application Data
(63) Continuation of application No. PCT/EP2022/083502, filed on Nov. 28, 2022.

(30) Foreign Application Priority Data
Nov. 29, 2021 (DE) 10 2021 1310279.5

(51) Int. Cl. A61B 17/122; A61B 17/00

(52) U.S. Cl. CPC A61B 17/1227; A61B 2017/00477; A61B 2017/00526

(57) ABSTRACT

A medical clip has two cooperating clamping arms and a biasing element. Each clamping arm is connected to an end of the biasing element by a connecting portion. The clamping arms are maximally proximate to one another, and may abut against one another, in a basic position of the clip, and are movable away from one another against the action of the biasing element from a basic position into an open position. The two connecting portions define a first circular cross section having a first diameter. The biasing element defines a second circular cross section having a second diameter smaller than the first diameter. The connecting portions taper toward the biasing element, such that conical transition regions are formed that define a cone angle. The cone angle can be at least 10°. A reshaping tool, a reshaping machine and a method can be used for producing a medical clip.

### CROSS-REFERENCE TO RELATED APPLICATIONS

This application is a continuation of International Application No. PCT/EP2022/083502, filed on Nov. 28, 2022, and claims priority to German Application No. 10 2021 131 279.5, filed on Nov. 29, 2021. The contents of International Application No. PCT/EP2022/083502 and German Application No. 10 2021 131 279.5 are incorporated by reference herein in their entireties.

### FIELD

The present disclosure relates to a medical clip, in particular in the form of an aneurysm clip, with two cooperating clamping arms and a biasing element having two ends, wherein each of the two clamping arms is connected to an end of the biasing element by way of a connecting portion, wherein the two clamping arms in a basic position of the clip are maximally proximate to one another, in particular abutting against one another, and are movable away from one another against the action of the biasing element from the basic position into an open position, wherein the two connecting portions define a first circular cross section having a first diameter, wherein the biasing element defines a second circular cross section having a second diameter, wherein the second diameter is smaller than the first diameter, and wherein the connecting portions taper in the direction toward the biasing element, such that conical transition regions are formed, which define a cone angle.

The present disclosure further relates to a reshaping tool, in particular swaging tool, for reshaping wire-shaped blanks, in particular to form medical clips, wherein the reshaping tool has a base body, which defines a longitudinal direction, on which base body at least one forming element is formed extending inclined relative to the longitudinal direction by an infeed angle.

The present disclosure also relates to a reshaping machine, in particular a rotary swaging machine.

The present disclosure further relates to a method for producing a medical clip, in particular in the form of an aneurysm clip, comprising the reshaping, in particular by rotary swaging, of a wire-shaped blank having a first circular cross section and a first diameter, wherein as a result of the reshaping, an intermediate portion having a second circular cross section and a second diameter is formed between two undeformed end portions of the blank, wherein as a result of the reshaping, at least one conical transition region between an undeformed end portion and the intermediate portion is formed.

### BACKGROUND

Medical clips of the kind described at the outset are used, in particular, in the form of aneurysm clips for the treatment of sacculations of hollow organs, for example on blood vessels. In order to achieve a larger elastic region of the basing element in such medical clips, it is known to alter the portion forming the biasing element of the medical clip in its structure by reshaping. Such cold hardening increases the yield strength in the metallic material from which the medical clip is formed. As a result, an increase in the opening width of a medical clip can be achieved. For reshaping, in particular, a rotary swaging process is used.

Known rotary swaging processes and reshaping tools and machines used for performing the same are known, e.g., from DE 203 09 632 U1. With such reshaping tools, in particular swaging tools, transition regions can only be formed having very small cone angles. In plunge and/or infeed rotary swaging with known reshaping tools, only limited deformation ratios are obtained, i.e. ratios between the second diameter and the first diameter. Furthermore, the size of the cone angle has an influence on the overall size of the aneurysm clip. The larger the cone angle is, the smaller the transition region between the biasing element and the respective connecting portion is. The shorter this transition region is, the closer the biasing element can be positioned relative to the clamping arms.

In the known reshaping tools, the forming elements are configured having a contour that defines a section of a hollow-cylindrical surface. Such a configuration of the reshaping tool limits the possibilities of reshaping in rotary swaging, in particular the deformation ratio, also referred to in the following as reduction ratio, thus being predetermined. However, the greater the deformation ratio is, the greater the quality losses are, because the material from which the blank is formed can flow into gaps between adjacent reshaping tools when the reshaping tools plunge into the blank for cold hardening, burrs thus being formed here. However, in particular in the case of medical clips, a formation of burrs in the transition region is not at all desirable. These may need to be removed in a further processing step, for example by machining or by grinding them smooth. However, this in turn affects the quality and strength of the medical clips.

### SUMMARY

It is therefore an object of the present disclosure to improve a medical clip, a reshaping tool, a reshaping machine, and a method for producing a medical clip in such a way that, in particular, compact medical clips of high quality can be formed.

This object is achieved, in accordance with the present disclosure, in a medical clip of the kind described at the outset in that the cone angle has a value of at least 10°.

Such cone angles cannot be formed using conventional reshaping tools. A cone angle of at least 10° reduces the transition region by nearly 50% compared to cone angles that are known from the prior art—caused by the symmetrical configuration of the forming elements on known reshaping tools—and which in the case of medical clips are at most about 7°. This allows more compact medical clips to be formed.

It is advantageous if the cone angle has a value of at least 15°. In particular, it may have a value of at least 20°. The larger the cone angle is, the shorter the transition region between the connection portions and the biasing element becomes. The biasing element, for example in the form of a coil spring, can thus move closer to the clamping arms. This is particularly advantageous for medical clips used in neurosurgery.

It is favorable if the first circular cross section defines a first cross sectional area, if the second circular cross section defines a second cross sectional area, and if a ratio of the second cross sectional area and the first cross sectional area is at most 0.7. In particular, the ratio may be at most 0.5. The specified ratio predetermines, in particular, the characteristic of the biasing element. The greater the deformation ratio de is, i.e. the ratio dA/A₀ between, on the one hand, the change dA of the cross sectional area, i.e. the difference between the cross sectional area A1 after the reshaping process and the cross sectional area A₀ before the reshaping process, and the cross sectional area A₀ on the other hand, the more the yield strength is increased, which makes the biasing element more elastic. Using known reshaping tools, medical clips can only be formed having cross sectional area ratios that are greater than 0.7. This corresponds to deformation ratios that are in the range of −0.3 to 0.

Preferably, the conical transition regions are formed exclusively by reshaping, in particular by rotary swaging. This has the advantage that in the production of the medical clips the transition regions can be formed in one single work step, namely by rotary swaging, for example. A reprocessing by grinding off burrs is then not necessary. This can be easily proven in a medical clip on the basis of geometric and microstructural properties. In particular, microstructure analyses can clearly determine whether the conical transition regions are formed exclusively by reshaping or whether they were additionally altered by being ground smooth or machined after reshaping.

The biasing element is favorably formed exclusively by reshaping, in particular by rotary swaging. Such a medical clip is of high quality. In addition, it is easy to manufacture, since the biasing element is able to be formed exclusively by reshaping. This work step defines the cross section reduction of a blank for forming the medical clip in the region of the biasing element, also referred to as the intermediate portion between end portions of the blank. This intermediate portion can then, for example, be wound into a spring. However, no rotary swaging process is used here.

It is advantageous if the conical transition regions are unground. Such a medical clip can be formed without burrs in the transition region, even without the need to grind the transition regions. This is possible, in particular, with cone angles as specified above. In known medical clips, such cone angles cannot be realized by reshaping alone, in particular rotary swaging. In addition, the formation of burrs in the transition region is practically unavoidable with conventional reshaping tools.

The biasing element is favorably unground. The biasing element can thus be formed with a high quality without negatively affecting the structure of the material by grinding or another machining process.

Particularly stable medical clips can be formed, for example, if they are of one-piece, in particular monolithic, configuration. Such a medical clip can then, for example, be formed from a blank by correspondingly reshaping different portions of the blank to form the clamping arms, the connecting portions, and the biasing element.

In order to move the clamping arms in a defined manner from the basic position into the open position and back from the open position into the basic position as a result of the biasing element, it is advantageous if the biasing element is configured in the form of a coil spring with at least one complete winding. The biasing element may also comprise, in particular, two, three, or more complete windings. In particular, a spring constant of the biasing element can be predetermined by the number of windings.

In accordance with a further preferred embodiment, provision may be made that clamping faces of the clamping arms define clamping face planes and that the clamping face planes are oriented in parallel to one another in the basic position. Such an embodiment of the clip makes it possible, in particular, to securely place the clip, for example on sacculations of hollow organs in a human or animal body, in particular known as so-called aneurysms.

The object stated at the outset is further achieved, in accordance with the present disclosure, in a reshaping tool of the kind described at the outset in that the at least one forming element is of asymmetrical configuration relative to a midplane of the base body containing the longitudinal direction.

Configuring a reshaping tool in this way makes it possible, in particular, to prevent or substantially prevent the formation of burrs on the workpiece during rotary swaging in the case of hollow-cylindrical form faces. The asymmetrical configuration makes it possible, in particular, to directly deform the burrs that typically form by correspondingly rotating the workpiece and reshaping tool. This is achieved, in particular, by the at least one forming element having a contour like conventional forming elements in known reshaping tools only over a portion of its forming face, said contour having an inner diameter that corresponds to the workpiece to be formed. However, due to the asymmetrical design of the forming tool, a region of the forming element can be designed such that a radius or a shape of the forming element cannot completely osculate the workpiece to be formed. By means of this region, with the reshaping tool and the workpiece that is to be processed being rotated accordingly relative to one another, material can then be successively deformed in such a way that no burrs form on the blank when it is deformed. Such a reshaping tool can be used, in particular, in plunge and/or rotary swaging and makes it possible, in particular, to achieve larger cone angles than in the past in transition regions, namely without forming burrs or other negative deformations that affect the quality of the workpiece. The infeed angle on the reshaping tool predetermines the cone angle in the transition region on the workpiece.

It is advantageous if the infeed angle has a value of at least 10°. In particular, it may have a value of at least 15°. Further in particular, it may have a value of at least 20°. With such reshaping tools, workpieces can then be formed in the described manner, for example exclusively by rotary swaging, without burrs in the region of the blank reduced in cross section, nor in the transition region.

In order to form the workpiece with a minimum cross section in a defined manner, it is favorable if a calibration region is formed on the base body, which extends in parallel to the longitudinal direction or defines the longitudinal direction.

For the reasons already explained, it is favorable if the calibration region is of asymmetrical configuration relative to the midplane. This allows the workpiece to be brought into its final desired shape by the calibration region.

In accordance with a further preferred embodiment, provision may be made that the forming element in a cross sectional plane perpendicular to the longitudinal direction defines a curved section line having a first end and a second end, that the curvature of the section line has a maximum value at the first end, and that the curvature of the section line has a minimum value at the second end. This configuration of the forming element makes it possible, in particular, to deform the workpiece in the region of the first end to a shape having a desired radius. A maximum value of the curvature corresponds to a minimum radius of the section line in the region of the first end. Thus, due to the section line extending in a curved manner as described, it is achieved that the reshaping tool successively osculates the workpiece when the reshaping tool and the workpiece successively come into contact with one another in different rotational positions relative to one another.

It is favorable if the curvature at the second end is zero. This means that the section line extends rectilinearly in the region of the second end. In particular, a planar region of the forming element can thus be created in order to deform the workpiece.

It is advantageous if the curvature of the section line decreases continuously commencing from the first end to the second end. Thus, a section line in the form of an involute can be created having a minimum radius in the region of the first end and a maximum radius in the region of the second end. The radius in the region of the first end is then never smaller than the radius of the finished workpiece.

Further, it may be advantageous if the curvature of the section line is constant commencing from the first end over an angular region of osculation defined relative to the longitudinal direction. As already mentioned, a region of the forming element can thus be created that corresponds to conventional reshaping tools. However, the difference to conventional reshaping tools is that the curvature is constant only in the angular region of osculation and then changes commencing from the angular region of osculation toward the second end.

It is advantageous if the curvature of the section line between the angular region of osculation and the second end is smaller than the curvature in the angular region of osculation. An asymmetrical reshaping element can thus be formed in a simple manner.

It is favorable if the curvature of the section line decreases commencing from the angular region of osculation toward the second end. In particular, it may decrease continuously. However, it may also decrease in one step, so that the angular region of osculation defines a first radius and the region between the angular region of osculation up to the second end of the section line defines a second radius that is greater than the first radius, and thus the curvature, which is smaller than the maximum curvature in the angular region of osculation.

In order to achieve a so-called “roof shape” of the reshaping element, it is advantageous if the curvature of the section line is constant commencing from the angular region of osculation up to the second end. Thus, the forming element can be formed by a combination of a partial region that corresponds to a conventional reshaping tool with a partial region that is planar.

In order to create a planar region between the angular region of osculation and the second end, it is advantageous if the curvature of the section line is zero commencing from the angular region of osculation up to the second end.

In accordance with a further preferred embodiment, provision may be made that the angular region of osculation relative to the longitudinal direction extends over a circumferential angle in a range of about 5° to about 90°. In particular, it may be in a range of about 10° to about 50°. Such angular regions of osculation allow optimum deformation of the workpiece.

Furthermore, it is favorable if the curvature at the first end of the section line of the infeed region increases, in particular continuously, in the direction toward the calibration region. In other words, this means that section lines in the infeed region that are closer to the calibration region have greater curvatures at the first end of the section line than those section lines that are further away from the calibration region. In this way, a workpiece can be successively reduced in its cross section by being repeatedly contacted by the reshaping tool and its forming element.

The object stated at the outset is further achieved, in accordance with the present disclosure, in a reshaping machine in that it comprises at least one of the reshaping tools described above.

In particular, it may include 2, 3, 4 or more reshaping tools. These are arranged, in particular, so as to be movable in the direction toward the longitudinal direction and can thus be moved toward the workpiece to deform shape and can be moved somewhat away from the workpiece to release same and to rotate the reshaping tools and the workpiece relative to one another about the longitudinal direction. The difference between the reshaping machine in accordance with the present disclosure and known reshaping machines is, in particular, the design of the reshaping tools, namely, in particular, their asymmetrical design.

The object stated at the outset is further achieved, in accordance with the present disclosure, in a method of the kind described at the outset in that the at least one conical transition region is configured having a cone angle of at least 10°. As already described at the outset, a shortened transition region has the advantage that the biasing element moves closer to the clamping arms and thus an overall size of the medical clip can be reduced.

It is advantageous if the intermediate portion is wound to form a biasing element while forming at least one complete winding. In particular, the winding can take place only when the clamping arms are deformed in the desired manner.

It is favorable if a respective first part of the two end portions are reshaped to clamping arms in such a way that the clamping arms extend commencing from free ends of the two end portions. For producing the medical clip, it is advantageous, in particular, if the clamping arms are formed before winding the biasing element, i.e. when the blank is still elongated after forming the intermediate portion reduced in cross section. The clamping arms can be formed, in particular, by pressing tools.

It is further advantageous if a respective second part of the two end portions is reshaped to a connecting portion in such a way that each connecting portion connects a clamping arm to an end of the biasing element, and if in a basic position of the clip the clamping arms are maximally proximate to one another, in particular abutting against one another, and are movable away from one another against the action of the biasing element from the basic position into an open position. The configuration of the connecting portions makes it possible, in particular, to adjust the medical clip in the desired manner. In particular, a biasing force exerted by the biasing element in the basic position can thus also be set. This can be predetermined in the desired manner by correspondingly deforming the connecting portions relative to the clamping arms and the biasing elements.

Preferably, the blank is reshaped using at least one of the reshaping tools described above to form the intermediate portion. This makes it possible, in particular, to directly deform the intermediate portion and the transition region in such a way that no burrs form and thus no reprocessing of the transition region and the intermediate portion is necessary. This is made possible by the asymmetrical design of the reshaping tools in a simple manner, said reshaping tools making cone angles in the transition region of more than 10° possible.

It is advantageous if, during the reshaping to form the intermediate portion, the blank and the at least one reshaping tool are rotated relative to one another about a longitudinal direction defined by the blank when they are out of engagement. As is typical in rotary swaging, a blank can thus be deformed in the desired manner by repeatedly opening and closing the reshaping tools.

A medical clip can be produced in a simple manner if it is reshaped using one of the reshaping machines described above.

In order to avoid damage to the clip, the at least one conical transition region is preferably configured unground. The transition region is thus formed only by rotary swaging.

Preferably, the at least one conical transition region is formed exclusively by reshaping, in particular exclusively by rotary swaging. This avoids further processing steps, which simplifies the production of a medical clip and also helps to minimize the cost of production.

Furthermore, the use of one of the methods described above for producing one of the medical clips described above is proposed.

### BRIEF DESCRIPTION OF THE DRAWINGS

The subsequent description of preferred embodiments serves in conjunction with the drawings for further explanation.

FIG. 1 shows a schematic perspective total view of an embodiment of a medical clip;

FIG. 2 shows a schematic depiction of a portion of a reshaping machine with three reshaping tools and a blank before the first plunge of the reshaping tools into the blank;

FIG. 3 shows a schematic view similar to FIG. 3, but wherein the reshaping tools of the reshaping machine are in contact with the workpiece;

FIG. 4 shows a section view along line 4-4 in FIG. 3;

FIG. 5 shows a section view along line 5-5 in FIG. 3;

FIG. 6 shows a schematic section view of an embodiment of a reshaping tool through its calibration region;

FIG. 7 shows a view similar to FIG. 6 of a further embodiment of a reshaping tool; and

FIG. 8 shows a view similar to FIG. 6 of a further embodiment of a reshaping tool.

### DETAILED DESCRIPTION

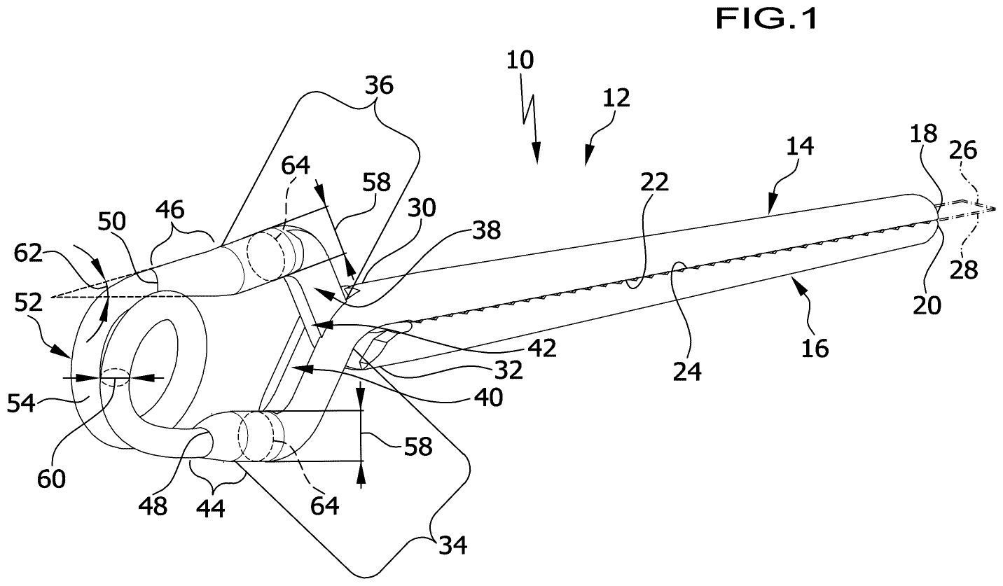

FIG. 1 shows schematically an embodiment of a medical clip, which is denoted as a whole with the reference numeral 10. It is configured in the form of an aneurysm clip 12.

The clip 10 comprises two cooperating clamping arms 14 and 16. The clamping arms 14 and 16 have free, distal ends 18 and 20.

Each clamping arm 14, 16 comprises a respective clamping face 22 and 24, which point toward one another. In a basic position depicted schematically in FIG. 1, the clamping arms 14 and 16 are maximally proximate to one another. In the embodiment depicted in FIG. 1, they abut against one another with their clamping faces 22 and 24.

The clamping arms 14 and 16 each define respective clamping face planes 26 and 28, which are oriented in parallel to one another in the basic position. In the embodiment depicted in FIG. 1, the clamping face planes 26 and 28 coincide.

Proximal ends 30 and 32 of the respective clamping arms 14 and 16 are each adjoined by a respective connecting portion 34 and 36. The connecting portions 34 and 36 are configured passing through one another in the embodiment depicted in FIG. 1. For this purpose, the connecting portion 36 comprises a male connection element 38, which passes through a female connection element 40 of the connecting portion 34 to form a box lock 42.

The connecting portions 34 and 36 taper in the direction toward ends 48 and 50 of a biasing element 52 while each forming a respective conical transition region 44 and 46. Thus, the two clamping arms 14 and 16 are each connected to a respective one of the ends 48, 50 of the biasing element 52 by way of the connecting portions 34 and 36.

The biasing element 52 is configured in the form of a coil spring 54 and has at least one complete winding. In the embodiment depicted in FIG. 1, the coil spring 54 comprises about 1.5 windings.

The clip is of one-piece, namely monolithic, configuration, made namely from a metallic, wire-shaped blank 56. The production of the clip 10 is explained in detail below.

The connecting portions 34 and 36 define a first circular cross section 64 having a first diameter 58. The biasing element 52 has a second circular cross section having a second diameter 60. The second diameter 60 is smaller than the first diameter 58.

The conical transition regions 44 and 46 define a cone angle 62, which has a value of at least 10°. In embodiments not shown, the cone angle 62 is at least 15° or at least 20°.

The first circular cross section 64, which defines the first diameter 58, defines a first cross sectional area due to its circular shape. The second circular cross section, which is defined by the second diameter 60, thus defines a second cross sectional area. In the case of the clip 10 schematically depicted in FIG. 1, a ratio of the second cross sectional area and the first cross sectional area is at most 0.7. In further embodiments, which are not depicted, the ratio of the second cross sectional area and the first cross sectional area is at most 0.5.

The conical transition regions 44 of the clip 10 are formed exclusively by reshaping, in particular by rotary swaging. The biasing element 52 is formed exclusively by reshaping, in particular by rotary swaging.

Both the conical transition regions 44 and the biasing element 52 are unground.

The production of the medical clip is explained in the following.

The medical clip 10 is formed from the blank 56 by reshaping, namely by rotary swaging. Known plunge and infeed rotary swaging methods can be used for this purpose.

The blank 56 is of wire-shaped configuration, i.e. in the form of an elongated piece of wire, and defines the first circular cross section 64 having the first diameter 58. By reshaping the blank 56, two undeformed end portions 66 are formed and an intermediate portion 68 extending between the end portions 66, which defines the second cross section having the second diameter 60.

As a result of the rotary swaging, a respective conical transition region 44 between the undeformed end portions 66 and the intermediate portion 68 is formed. The cone angle 62 defined by the conical transition region is at least 10°.

After forming the intermediate portion 68 by rotary swaging, a respective portion of the end portions 66 is reshaped to one of the clamping arms 14 and 16, namely in such a way that the clamping arms 14, 16 extend commencing from free ends of the two end portions 66. Thus, the free ends 18 and 20 form the free ends of the two end portions 66.

In a next step, the intermediate portion 68 is wound, forming at least one complete winding, to form the biasing element 52, namely to form the coil spring 54.

In a next step, those portions of the two end portions 66 that have not been reshaped to the clamping arms 14, 16 are each reshaped to a respective connecting portion 34 and 36, namely in such a way that each connecting portion 34, 36 connects one of the clamping arms 14 or 16 to one of the ends 48, 50 of the biasing element 52 and that in the basic position of the clip 10 the clamping arms 14, 16 are maximally proximate to one another and are movable away from one another against the action of the biasing element 52 from the basic position into an open position. The box lock 42 provided as an example in FIG. 1 in the depicted embodiment can then be formed in this step.

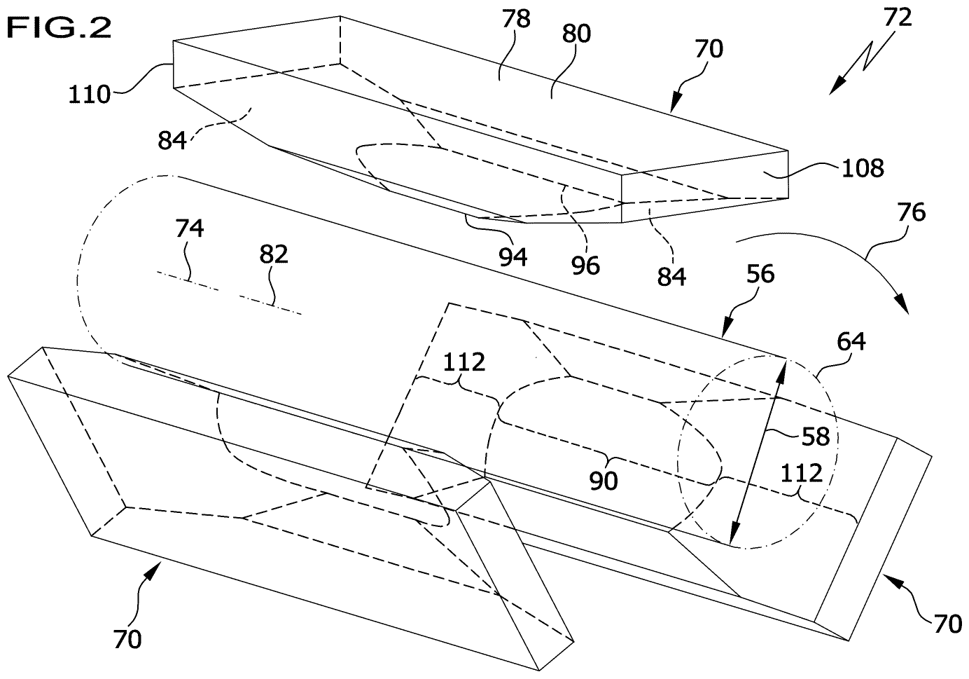

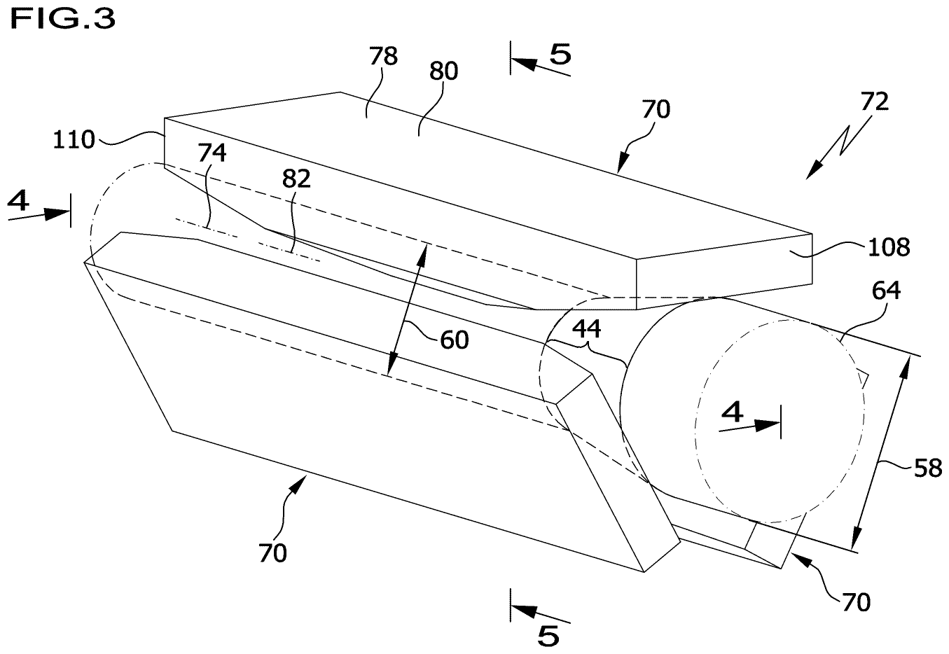

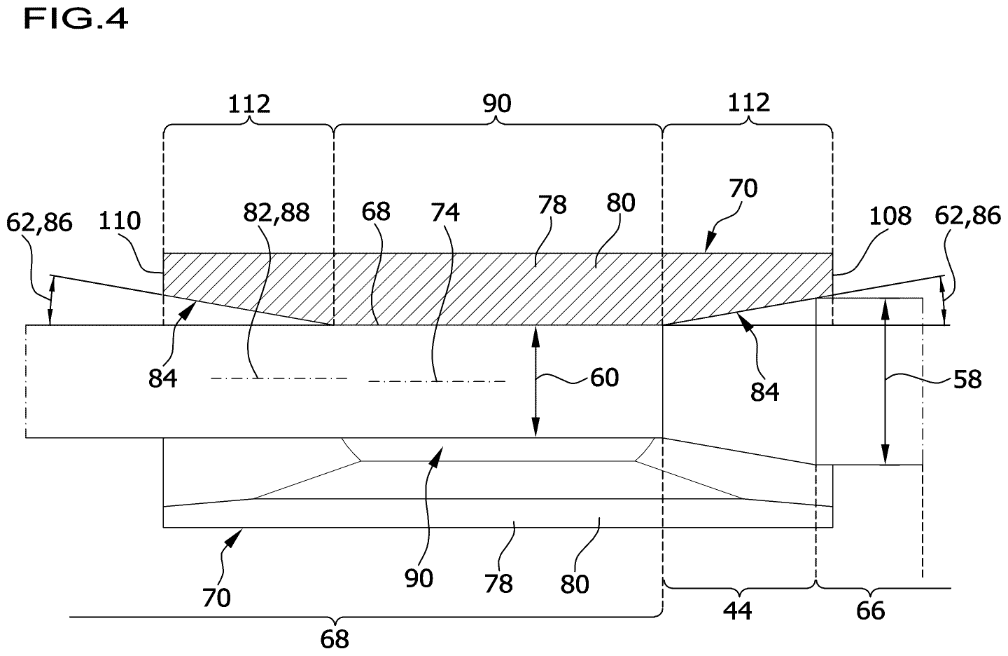

In the production of the clip 10, to form the intermediate portion 68, the blank 56 is reshaped with one or more reshaping tools 70 or with a reshaping machine 72, which is schematically depicted in FIGS. 2 to 5, namely with the three reshaping tools 70 comprised by said reshaping machine. In alternative embodiments, the reshaping machine 72 may also comprise 2, 4, or 5, in particular identical, reshaping tools 70.

In the production of the clip 10, the conical transition regions 44 are of unground configuration. This means that they are not reground or otherwise reprocessed after being reshaped by rotary swaging. This is also not necessary when using the reshaping tools 70 described in more detail below, unlike in the case of the reshaping tools known from the prior art. Thus, both the conical transition region 44 and the intermediate portion 68 can be formed exclusively by reshaping, namely exclusively by rotary swaging.

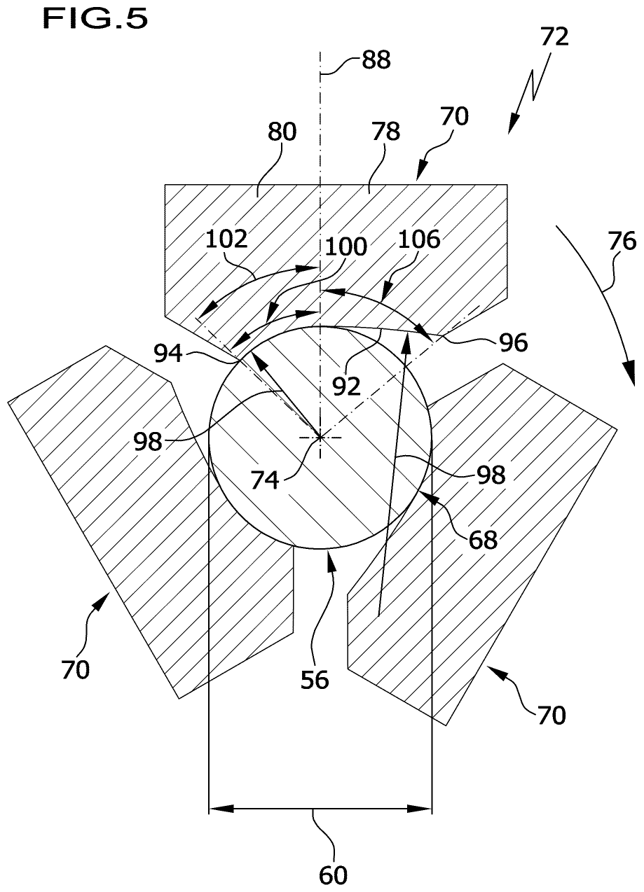

In order to avoid a formation of burrs in the regions between adjacent reshaping tools 70 when the reshaping tools 70 plunge into the blank 56, the reshaping tools 70 during reshaping for forming the intermediate portion 68 are rotated relative to the blank 56 about a longitudinal direction 74 defined by the blank 56 when they are out of engagement, i.e. not in contact with one another. The direction of rotation, assuming a fixed blank 56, is shown schematically by the arrows 76 in FIGS. 2 and 5.

The blank 56 is made of a biocompatible metal, for example an instrument steel or titanium.

In order to form the conical transition regions 44 and 46 in the manner described, the reshaping tools 70 are used, which are configured in the form of swaging tools 78. Using said swaging tools, the wire-shaped blanks 56, as described, can be reshaped in a first step in order to then in further steps form a medical clip 10 with this already processed workpiece.

Schematically depicted in FIGS. 2 to 5 is a first embodiment of a reshaping tool 70. It comprises a base body 80, which defines a longitudinal direction 82 that runs parallel to the longitudinal direction 74 or also coincides therewith.

In the case of the reshaping tools 70 in FIGS. 2 to 5, formed on the base body 80 are two forming elements 84, which are inclined relative to the longitudinal direction 82 by an infeed angle 86.

The special feature of the forming elements 84 in comparison to forming elements of reshaping tools known from the prior art is that they are of asymmetrical configuration relative to a midplane 88 of the base body 80 containing the longitudinal direction 82. The exact shape is described in more detail below.

In the embodiment depicted in FIGS. 2 to 5, the infeed angle 86 is at least 10°. In further embodiments, it may be at least 15° or even at least 20°.

Furthermore, a calibration region 90 is formed on the base body 80, said calibration region extending in parallel to the longitudinal direction 82 or defining the longitudinal direction 82. The calibration region 90 is of asymmetrical configuration relative to the midplane 88.

In particular, the forming element 84 serves to characterize the reshaping tool 70. Said forming element has in a cross sectional plane perpendicular to the longitudinal direction 82 a curved section line 92, also referred to as a boundary line, which is shown schematically in FIG. 5. The section line 92 has a first end 94 and a second end 96. The forming element 84 is concavely curved in the direction toward the blank 56. Correspondingly, the section line 92 is concavely curved.

The curvature of the section line 92 has its maximum value at the first end 94. This means that a radius 98 of the forming element 84 has the smallest value at the first end 94. The curvature of the section line 92, on the other hand, has a minimum value at the second end 96. Thus, a radius in the region of the second end 96 is maximum.

In the embodiment of FIGS. 2 to 5, the curvature of the section line 92 decreases commencing from the first end 94, but not completely continuously. Rather, the curvature of the section line 92 is constant commencing from the first end 94 over an angular region of osculation 100 defined relative to the longitudinal direction 82. The angular region of osculation 100 extends, relative to the longitudinal direction 82, over a circumferential angle 102 that is in a range of about 5° to about 90°. In the embodiment of FIGS. 2 to 5, the circumferential angle 102 of the angular region of osculation 100 is about 50°.

In the embodiment of the reshaping tools 70 in FIGS. 2 to 5, the curvature of the section line 92 is constant commencing from the angular region of osculation 100 up to the second end 96. Thus, a radius 98 in this portion is greater than the radius 98 in the angular region of osculation 100.

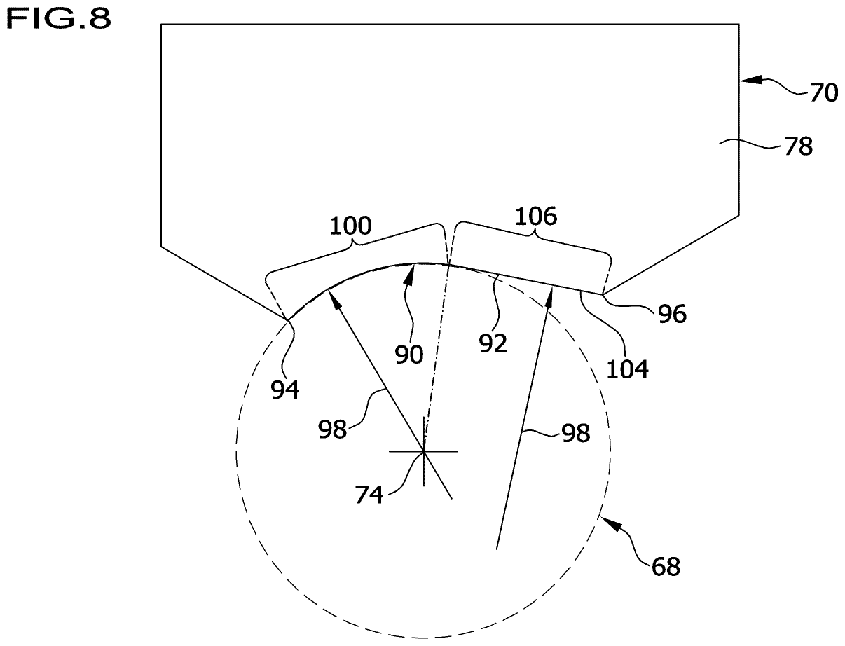

The radius 98 in the region between the angular region of osculation 100 up to the second end 96 may be infinitely large. Thus, in this case, the curvature in the portion between the angular region of osculation 100 and the second end is zero. This means that a planar portion 104 of the forming element 84 is created here. Such a reshaping tool 70 is schematically depicted in FIG. 8.

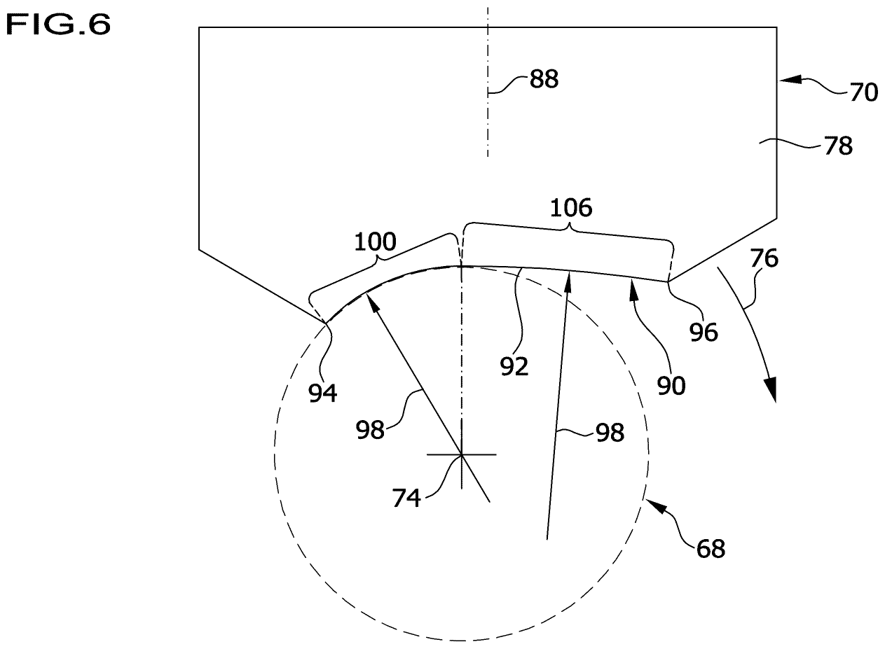

FIG. 6 shows schematically a reshaping tool 70, which corresponds in its basic structure with the reshaping tools 70 of FIGS. 2 to 5. Here, an angular region of osculation 100 is thus formed by a hollow-cylindrical wall face in the calibration region 90 having a radius 98, which corresponds to half the second diameter of the intermediate portion 68, and having an opening region 106, which extends between the angular region of osculation 100 and the second end 96. The two radii 98 differ from one another. Furthermore, the angular region of osculation 100 and the opening region 106 transition one into the other without kinking.

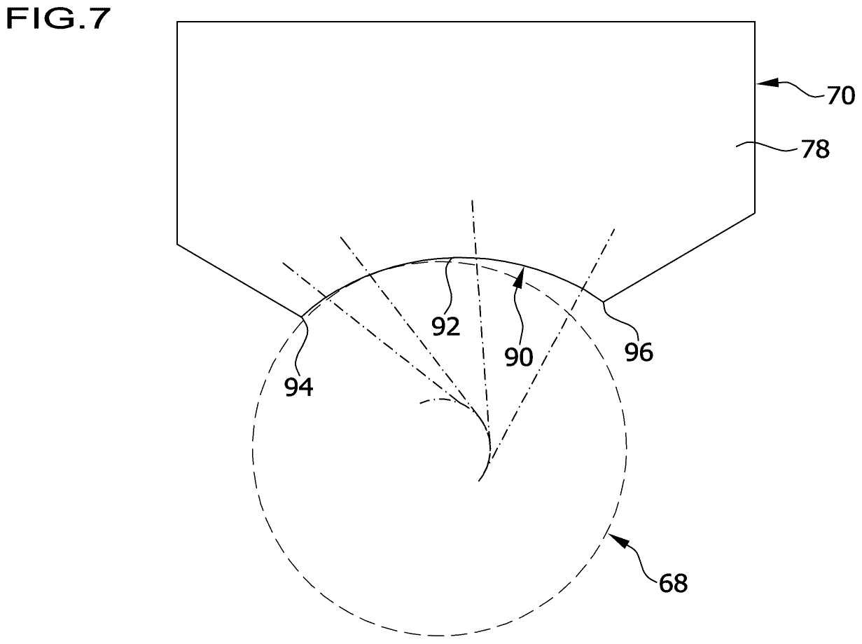

A further embodiment of a reshaping tool 70 is schematically depicted in FIG. 7. Here, the curvature of the section line 92 decreases continuously commencing from the first end 94 toward the second end 96. Thus, the section line 92 can be described as an involute.

The reshaping tools 70 are further configured in such a way that the curvature at the first end 94 of the section line 92 of the infeed region 112 increases in the direction toward the calibration region 90. This means in other words that at cross sections of the reshaping tool 70 perpendicular to the longitudinal direction 82 commencing from mutually averted ends 108 and 110 of the base body 80, the curvature at the first end 94 of the respective section line 92 increases in the direction toward the calibration region 90. The infeed region 112 is that region in which the forming element 84 encloses the infeed angle 86 with the longitudinal direction 82. Due to this configuration of the infeed region 112 on both sides of the reshaping tool 70, the wire-shaped blank 56 can be reshaped between the end portions 66 to form the intermediate portion in the described manner.

With the described novel design of the reshaping tools 70, the disadvantages known from the prior art can be avoided. One such disadvantage is, in particular, that the forming elements, which are symmetrical in cross section, also referred to as dies, greatly limit the achievable radial travel of the reshaping tools 70 and the infeed angle that can be set.

The special asymmetrically designed contour of the forming element 84, its section line 92 perpendicular to the longitudinal direction 82 having no symmetry between the ends 94 and 96, prevents the formation of burrs both on the intermediate portion and at the conical transitions regions 44.

In the construction of the reshaping tools 70, the relative rotational movement between the blank 56 and the reshaping tools 70 during the rotary swaging process is taken into account. Due to the special shape of the forming element 84, in the region of the material moving into the opening region 106 due to the described rotational movement between the blank 56 and the reshaping tool 70, the forming element 70 is open such that a penetration of a tool edge of the reshaping tool 70, namely the tool edge defined by the second end 96 of the section line 92, into the blank 56 is prevented as deformation ratios increase, i.e. as the amount of material that is deformed increases. For example, in the case of a contour of the forming element 84 that is planar on one side or that has a defined larger radius, the angular region of osculation with a constant radius, which as a high degree of osculation, i.e. a radius, serves to determine the radius of the finished rotary swaged workpiece.

The proposed asymmetrical geometry of the reshaping tools 70 allows greater deformation ratios and greater infeed angles 86 compared to the prior art. Furthermore, the asymmetrical shape of the forming elements 84 prevents the formation of wings or polygons caused by reshaping tools known from the prior art, so that an osculation with a value close to 1 is possible. This means that radii of the workpiece, in particular of the intermediate portion 68, and of the section line 92 in the angular region of osculation 100 or near the first end 94 are identical.

Workpieces that are rotary swaged with the asymmetrical reshaping tools 70 can be identified by their geometric and microstructural properties. Workpieces produced in this way have a deformation ratio or reduction ratio and an infeed angle that lie outside the limits known from the prior art.

With the described method, a blank 56 can thus be rotary swaged in a first step to form two end portions 66 and an intermediate portion 68 arranged therebetween. The conical transition regions 44 formed with the proposed reshaping tools 70 and the intermediate portion 68 do not need to be reworked and also have a high quality surface without burrs and formed polygons.

### Reference Numeral List

10 medical clip
12 aneurysm clip
14 clamping arm
16 clamping arm
18 free end
20 free end
22 clamping face
24 clamping face
26 clamping face plane
28 clamping face plane
30 proximal end
32 proximal end
34 connecting portion
36 connecting portion
38 connection element
40 connection element
42 box lock
44 conical transition region
46 conical transition region
48 end
50 end
52 biasing element
54 coil spring
56 blank
58 first diameter
60 second diameter
62 cone angle
64 first cross section
66 end portion
68 intermediate portion
70 reshaping tool
72 reshaping machine
74 longitudinal direction
76 arrow
78 swaging tool
80 base body
82 longitudinal direction
84 forming element
86 infeed angle
88 midplane
90 calibration region
92 section line
94 first end
96 second end
98 radius
100 angular region of osculation
102 circumferential angle
104 planar portion
106 opening region
108 end
110 end
112 infeed region

1. A medical clip comprising:
two clamping arms that cooperate; and
a biasing element having two ends,
each of the clamping arms being connected to an end of the biasing element by way of a connecting portion,
the clamping arms are maximally proximate to one another in a basic position of the medical clip and are movable away from one another against an action of the biasing element from the basic position into an open position,
the connecting portions defining a first circular cross section having a first diameter,
the biasing element defining a second circular cross section having a second diameter,
the second diameter being smaller than the first diameter, and
the connecting portions tapering toward the biasing element such that conical transition regions are formed, the conical transition regions defining a cone angle having a value of at least 10°.

2. The medical clip according to claim 1, wherein the cone angle has a value of at least 15°.

3. The medical clip according to claim 1, wherein the first circular cross section defines a first cross sectional area, the second circular cross section defines a second cross sectional area, and a ratio of the second cross sectional area and the first cross sectional area is at most 0.7.

4. The medical clip according to claim 1, wherein:
a) the conical transition regions are produced exclusively by reshaping, and/or
b) the biasing element is produced exclusively by reshaping.

5. The medical clip according to claim 1, wherein:
a) the conical transition regions are unground, and/or
b) the biasing element is unground.

6. The medical clip according to claim 1, wherein:
a) the medical clip is of a one-piece monolithic construction, and/or
b) the biasing element is configured as a coil spring with at least one complete winding.

7. The medical clip according to claim 1, wherein clamping faces of the clamping arms define clamping face planes, and the clamping face planes are oriented in parallel to one another in the basic position.

8. A reshaping tool for reshaping wire-shaped blanks, the reshaping tool comprising:
a base body defining a longitudinal direction; and
at least one forming element formed on the base body and extending inclined relative to the longitudinal direction by an infeed angle,
the at least one forming element having an asymmetrical configuration relative to a midplane of the base body containing the longitudinal direction.

9. The reshaping tool according to claim 8, wherein a calibration region is formed on the base body, the calibration region extending in parallel to the longitudinal direction or defining the longitudinal direction.

10. The reshaping tool according to claim 8, wherein the forming element defines a curved section line in a cross sectional plane perpendicular to the longitudinal direction, the curved section line having a first end and a second end, wherein a curvature of the curved section line has a maximum value at a first end of the curved section line, and wherein the curvature of the curved section line has a minimum value at a second end of the curved section line.

11. The reshaping tool according to claim 10, wherein the curvature of the curved section line is constant commencing from the first end of the curved section line over an angular region of osculation defined relative to the longitudinal direction.

12. The reshaping tool according to claim 11, wherein the curvature of the curved section line, commencing from the angular region of osculation:
a) decreases toward the second end, or
b) is constant up to the second end.

13. The reshaping tool according to claim 11, wherein the angular region of osculation relative to the longitudinal direction extends over a circumferential angle in a range of about 5° to about 90°.

14. The reshaping tool according to claim 10, wherein the curvature at the first end of the curved section line of an infeed region increases in a direction toward a calibration region.

15. A reshaping machine comprising at least one reshaping tool according to claim 8.

16. A method for producing a medical clip, the method comprising the steps of:
reshaping a blank that is wire-shaped, the blank having a first circular cross section and a first diameter;
forming an intermediate portion having a second circular cross section and a second diameter between two undeformed end portions of the blank; and
forming at least one conical transition region between an undeformed end portion and the intermediate portion, wherein the at least one conical transition region is configured having a conical angle of at least 10°.

17. The method according to claim 16, wherein:
a) the intermediate portion is wound forming at least one complete winding to form a biasing element, and/or
b) a respective first part of the two end portions is reshaped to clamping arms in such a way that the clamping arms extend commencing from free ends of the two end portions.

18. The method according to claim 16, wherein the blank is reshaped with at least one reshaping tool to form the intermediate portion.

19. The method according to claim 16, wherein the blank is reshaped with a reshaping machine to form the intermediate portion.

20. The method according to claim 16, wherein the at least one conical transition region is formed:
a) unground and/or
b) exclusively by reshaping.
